(19) United States
(12) Patent Application Publication
(10) Pub. No.: US 2005/0265938 A1
(43) Pub. Date: Dec. 1, 2005

(54) Novel color cosmetic compositions

(76) Inventors: Isaac David Cohen, Brooklyn, NY (US); Jennifer Oko, Stony Brook, NY (US); Isabelle Afriat, New York, NY (US)

Correspondence Address: Karen A. Lowney, Esq.;ESTEE LAUDER COMPANIES, Ste. 345 South, 155 Pinelawn Road, Melville, NY, 11747, US

(21) Appl. No.: 11/133,558

(22) Filed: May 20, 2005

Related U.S. Application Data
(60) Provisional application No. 60/574,172, filed on May 25, 2004.

(51) Int. Cl. A61K 7/021

(52) U.S. Cl. 424/63

(57) ABSTRACT

The invention provides unique cosmetic compositions containing at least two different pigment types and a carrier, the pigments of different types being physically separated from each other in individual capsules within the carrier. Compositions of the invention provide vibrant color to the skin, but without the ashiness typical of compositions containing traditional pigment grinds.

### FIELD OF THE INVENTION

The invention relates to the field of cosmetics. More specifically, the invention relates to a color cosmetic having a unique configuration of contained pigments, resulting in unusual optical effects.

### BACKGROUND OF THE INVENTION

The gold standard for a color cosmetic is one that confers to the wearer an enhanced appearance of the features to which it is applied, while at the same time making it appear as if it were the wearer's natural beauty that is being viewed. Over the hundreds of years in which cosmetics have been used, women (or men) have gradually turned their preference from the fashion of very heavily pigmented, opaque formulations, which more hid the user's features than enhanced them, to the color products which augment, but don't obfuscate the user's natural appearance. While there is still a market for the opaque formulations, such as concealers, that physically cover and hide skin flaws, such products find relatively limited use, and there is an ongoing search for makeup formulations that will confer the desired natural appearance while still improving the overall look of the wearer. One problem in achieving this is the nature of the traditional pigments themselves. Typically most color cosmetics are largely based on metallic oxides, such as iron oxides and titanium dioxide. These materials, coming in shades of red, brown, black, yellow and white, all tones found naturally in skin, when ground together in the correct proportions, can confer a variety of skin colors to a formulation; because of their relative opacity, they are particularly useful in concealment, and therefore have in the past been highly favored for use in foundations and concealers. The creation of a makeup without the use of metal oxides is virtually unthinkable even today. However, with the trend toward less opacity being favored, new ways of exploiting their useful properties, while downplaying their undesirable opacity and ashiness, are constantly being sought. The goal of most makeups today is to provide an effective concealer of flaws and unevenness without conveying the look of a mask. The present invention provides a novel approach to solving this problem.

### SUMMARY OF THE INVENTION

The present invention relates to a cosmetic composition containing at least two different pigments and a carrier, the pigments of different types being physically separated from each other in the carrier by encapsulation of the pigments, each capsule containing a single type of pigment. The invention also relates to a method of making a cosmetic composition, containing at least two different pigments and a carrier, the method comprising the steps of (a) preparing one extender for each different pigment, and (b) blending each extender into the carrier without grinding the pigments together, whereby there is no physical contact in the carrier between the different types of pigments. The cosmetic of the invention provides excellent coverage, and yet appears sheer and natural on the skin.

### DETAILED DESCRIPTION OF THE INVENTION

The cosmetics of the present invention take their inspiration from the artistic Impressionist movement. Essentially, this approach to painting relied on the presentation on canvas of individual dots or short strokes of color, which when viewed at a distance communicates an immediate impression of a cohesive image, but which also conveys an image of ethereal beauty and illumination which is otherwise not achievable with typical intensely colored but opaque oil paint pigments. In like manner, the present composition provides a color cosmetic in which the whole is considerably more than the sum of its individual parts: the makeup when applied is a very effective concealer, utilizing the useful camouflaging qualities of the metallic oxides to great effect, yet at the same time conveying to the viewer a look that is sheer and natural.

The cosmetic of the invention employs traditional pigments in a way that has not previously been employed in color cosmetics. The usual approach to producing color cosmetics is to grind the metal oxide pigments together, providing a uniform mix of the pigments in the product. The pigments themselves in the final product are indistinct, conferring an opaque, homogeneous, skin colored shade to the composition. In contrast, the compositions of the present invention do not utilize grinding of the pigments; instead, the pigments are added to the composition in such a way that the individual colors remain separate and distinct in the final product and on the skin. The result of this unique preparation is vibrant color which achieves the desired effect of masking flaws or evening skin tone, but which to the viewer appears natural, uniform and free of ashiness.

The composition of the invention achieves this result by ensuring that each individual color pigment is encapsulated separately from the pigments of other types. In other words, black iron oxide pigment is the only pigment found within its capsule, and red iron oxide is the only pigment found within its capsule. There is no direct physical contact between pigments of different types in the final composition. This encapsulation can be achieved in a variety of ways. In one embodiment, each individual pigment can be separately physically encapsulated in any manner which is known in the art, for example, a silica encapsulated black pigment, or a silica encapsulated red pigment, can be combined by simple, gentle mixing of the individual encapsulated pigments into the cosmetic carrier. Alternately, the pigments can be encapsulated in individual micelles within the composition, each micelle, and therefore each individual pigment, remaining physically separate from the others in the composition. Although not in the strict sense encapsulation, the latter approach effectively achieves the same result by permitting the distinct colors to remain apart in the carrier. Thus, for purpose of the present specification and claims, “encapsulation” or “capsules” will be understood to encompass not only traditional encapsulation in which a pigment may be contained within a physical capsule, but also any method of preparation of the composition which results in the physical separation of the pigments within the carrier, such as containment of individual pigments in distinct micelles within the carrier.

The composition of the invention can be in any form typical for color cosmetics, e.g., liquid, solid or powder, and may be water-containing, e.g., an emulsion (oil or silicone in water, or water in oil or silicone, or multiple emulsions) or an anhydrous formulation. Except as discussed in more detail as follows, all formulations are prepared in the same fashion as standard products of this type, and techniques for preparation of various carrier forms are well known in the art, and described generally in a number of sources, including for example, Remington's Pharmaceutical Sciences, or Harry's Cosmeticology, the contents of which are incorporated herein by reference. The claimed formulations differ from standard, however, in that a key to success in achieving the desired result is to formulate the composition in such a way as to (a) achieve the separate encapsulation of the different types of pigments, so that only one pigment type occurs in each capsule; and (b) ensure no merging or agglomeration of the individual color capsules once they are incorporated into the carrier. Component (a) of the process can be achieved in a number of different ways. As already noted above, one relatively simple method of achieving this is to utilize pigments that have already been individually encapsulated, i.e., the pigment or colorant is itself an individual chemical shell, or it is surrounded by a chemical shell, so that there is a single type of pigment within each capsule, and there is substantially no physical merging of pigments or colorants of different colors. A variety of different colorants of this type may be employed, for example, individual glass beads of different colors constituting their own chemical shell, or pigments encapsulated in a chemical shell, such as silica, that may or may not be further surface treated to render them dispersible in the phase of choice, i.e., hydrophilic treatment for use in a water phase, and hydrophobic treatment for use in an oil or silicone phase. A one specific example of this type of pigment is a silica-encapsulated pigment, available from Dominion Color Corporation (Toronto, Ontario, Canada). One preferred pigment of this type is about 60% metal oxide and about 40% silica. Such encapsulated pigments are particularly useful in an anhydrous formulation, such as a powder product, or in any oil phase, such as an anhydrous hot pour, or the oil phase of an emulsion.

An alternate mechanism for achieving encapsulation of the pigment is to envelop the pigment particle in a micelle, i.e., in an oil or water droplet that comprises the dispersed phase of an emulsion. This can be achieved by preparing individual emulsions for each of the pigments to be used in the final formula, i.e., preparing full formula extenders for each individual pigment. In this methodology, the pigments are simply incorporated into the internal phase of the emulsion, i.e., the water phase of a water-in-oil emulsion, or the oil phase of an oil-in-water emulsion. Each individual pigment emulsion is then subsequently incorporated into the carrier emulsion. Each pigment type thus remains separated from the other pigment types by the boundaries of the individual micelles. The pigments used in this encapsulation method can be employed without any surface treatment; however, it is preferred in this scenario, in order to prevent agglomeration of the individual micelles to employ a pigment that has been surface treated in a manner appropriate to the composition of the phase it is to be incorporated in. For example, pigments to be incorporated into an oil phase are preferably treated, partially or fully, with a hydrophobic coating, such as magnesium myristate, fluorinated coatings, or silicone-based coatings, such as methicone, dimethicone, alkyl silanes, isododecane and the like, which are particularly useful when used in a silicone phase. Similarly, pigments to be used in a water phase are preferably hydrophilically treated. Examples of useful hydrophilic treatments are dimethicone copolyol or lecithin. Examples of useful pigments having a hydrophilic coating are the AQ Series of pigments available commercially from Cardre.

The second aspect of achieving the unique formulation of the invention ensuring that the capsules remain separate once incorporated into the carrier. This can be accomplished in different ways. One important approach to achieving this is by the manner in which the capsules are incorporated. In the preparation of a traditional color cosmetic, in order to achieve a homogeneous incorporation of the ground pigments, the formulation is subject to relatively high shear, i.e., from about 800 to about 1800 rpm on a three-prop mixer, preferably about 1200 to about 1800 rpm, mixing to ensure uniformity. This is crucial to achieving the uniform distribution of the ground pigments throughout the carrier; the merger of individual micelles, which is inevitable in such processing of an emulsion is irrelevant, first because, in most cases of traditional makeups, pigments are usually incorporated into a continuous, and not dispersed phase and second, even if the pigments are in the dispersed phase, they have already been commingled. However, in the present compositions, such vigorous mixing is undesirable, because it will potentially cause the coalescence of individual micelles, thereby partially or completely obliterating the desired effect. A similar effect can occur with the physically encapsulated pigments, where the capsule can be broken and intermixing of pigment particles can then occur. Thus, in the present compositions, the pigments must be gently mixed into the carrier emulsion.

Gentle incorporation can be achieved in a variety of ways. With a powder formulation, for example, all powder components can be initially ground together, and then the individual encapsulated pigments mixed at low speed, i.e., at about 500 to about 800 rpm, into the rest of the powder components. Alternately, individual encapsulated pigments can be incorporated into separate full formula extenders, and then combined to form the final product. The powder can then remain loose, or can be pressed in accordance with procedures typically used for this purpose. For a liquid oil phase, e.g., a hot pour or an emulsion, the encapsulated pigments can simply be directly added to the oil phase.

If an emulsion is to be prepared, a preferred methodology is to prepare separate formula extenders for each pigment to be used thereby forming several individual emulsions, each containing a single type of pigment in its dispersed phase. Alternately, individual intended dispersed phases can be prepared, each containing a different pigment. The extenders, or the intended dispersed phase, are then added individually to the pre-made carrier emulsion or to the pre-made intended continuous phase, and as with the powder, gently mixed, at a speed of no greater than about 500 to about 1200 rpm (higher viscosities requiring speeds at the higher end of the range), to avoid coalescence of the micelles that have already formed in the extenders or which are formed upon the addition of the intended dispersed phase to the continuous phase.

Although the very moderate mixing speed can be effective, on its own, in maintaining separation of differently pigmented micelles, it is ordinarily preferable to incorporate one or more emulsifiers into one or more of the phases to maintain the stability of the separate droplets over prolonged periods. In general, the emulsifier will be chosen in accordance with the nature of the emulsion, i.e., a water-in-oil emulsifier, with an HLB of from 2-6 will be used in a water-in-oil emulsion, and an oil-in-water emulsion, having an HLB of greater than 6 will be used in an oil-in-water emulsion. Examples of useful water-in-oil emulsifiers include, but are not limited to, sorbitan derivatives such as sorbitan laurate and sorbitan palmitate; alkoxylated alcohols such as laureth-4; hydroxylated derivatives of polymeric silicones, such as dimethicone copolyol; alkylated derivatives of hydroxylated polymeric silicones, such as cetyl dimethicone copolyol; glyceryl esters such as polyglyceryl-4 isostearate; beeswax derivatives such as sodium isostearoyl-2 lactylate; and mixtures thereof. Examples of useful oil-in-water emulsifiers include but are not limited to sorbitol derivatives, such as sorbitan monolaurate and polysorbate 20; ethoxylated alcohols such as laureth-23, ethoxylated fatty acids such as PEG-1000 stearate; amidoamine derivatives such as stearamidoethyl diethylamine; sulfate esters such as sodium lauryl sulfate; phosphate esters such as DEA cetyl phosphate; fatty acid amine salts such as TEA stearate; and mixtures thereof. Additional examples of appropriate surfactants or emulsifiers can also be found in McCutcheon's Emulsifiers & Detergents, 2004 edition, the contents of which are incorporated herein by reference. In a preferred embodiment, in which the emulsion is a water-in-silicone emulsion, the emulsifier used is an emulsifying silicone elastomer. Emulsifying silicone elastomers are typically crosslinked organopolysiloxanes with at least one oxyalkylene unit. Compounds of this type are disclosed in U.S. Pat. Nos. 5,412,004; 5,837,793; 6,103250; 5,919,437, and 5,811,487, the contents of which are incorporated herein by reference. Commercially available examples of emulsifying silicone elastomers include dimethicone/dimethicone copolyol crosspolymers, such as are available from Shin-Etsu or Dow Corning. A particularly preferred crosspolymer is sold by Shin-Etsu under the name KSG-210. The amount of emulsifier used in the emulsion will depend on the emulsifier chosen and the composition of the product, but as a general guideline, the emulsifier will be present in an amount of from about 0.5 to 15%, preferably about 2 to about 10%.

It may also be desired to add further stabilizing materials to the composition. For example, viscosity enhancers may aid in stabilizing an emulsion. These enhancers can be used in one or more phases of the emulsion, water soluble or miscible materials being used in a water phase, and oil soluble or oil miscible materials being used in the oil phase. Examples of useful viscosifiers include but are not limited to acrylates crosspolymers and copolymers, carbomers, non-emulsifying silicone elastomers, polysaccharides or gums, cellulosics, sulfonic acid polymers, acrylamide polymers, clays, and the like. Additional viscosifying agents are well-known, and can be found, for example, in the International Cosmetic Ingredient Dictionary and Handbook.

The relative proportions of water phase to oil phase are not critical, and can be in the range of from about 10:90 to 90:10, preferably in the range of about 30:70 to 70:30, and more preferably from about 30:70 to about 50:50, depending upon the type and quality of emulsion desired. Generally speaking, the larger the percentage of water, the thicker and creamier the product will be, and products with less water will be more fluid.

An optional feature of the emulsion compositions of the present invention is the control of the size of the dispersed micelles. Generally speaking, the micelle size range will be in the range of from about 1 to about 400μ.. Although formulas containing micelles in any of these size ranges will perform as described, the formulations containing the larger size micelles, i.e., averaging in the 100-400μ range, tend to have a somewhat grey appearance. Although this does not detract from performance on the skin, it presents a less attractive appearance in a package. It is therefore preferred that emulsion formulations contain micelles that average less than 100μ in diameter, preferably in the range of about 1 to about 60μ range, and more preferably in the range of about 5 to about 20μ. Those formulations containing smaller micelles tend to have a more beige appearance, thus resembling more typical makeup in its packaged form. The size of the micelles can be controlled by the amount of energy put into the system, i.e., the speed and/or shear with which the product is processed. Lower speed and shear result in larger micelles, and greater speed and shear result in smaller micelles.

The concept of the invention can be applied to any type of pigment The pigments employed in the invention are preferably metal oxides, in particular, iron oxides (red, black, yellow, brown), titanium dioxide (white), zinc oxide, chrome oxide (green), but the invention can also be applied to other pigment types, such as organic dyes, interference pearls and powders that confer an optical effect, such as soft focus powders or bismuth oxychloride. When utilizing pigments other than the metal oxides, it will be important to control the particle size, e.g., use of only smaller interference pearls, i.e., about 1 to about 10μ, and, with respect to the organic dyes, to present them in a way that they will not run, e.g., a hydrophobic coating or encapsulation. The amount of pigments used is not critical, and will depend upon the shade and effect desired. Generally, however, the overall amount of pigment will be in the range of from about 0.1 to about 30% by weight of the composition, preferably about I to about 15%..

The compositions of the invention can be used as any type of color cosmetic, e.g., a foundation, concealer, bronzer, blush, eyeshadow, eyeliner, mascara, lipstick, lip gloss, or lipliner, although the greatest advantage is observed when the product is a facial or body foundation. The technology can also be applied to skin care compositions in which a particular visual effect is desired but is not the main focus of the product, e.g., a tinted moisturizer or sunscreen.

The invention is further illustrated by the following non-limiting examples:

### EXAMPLE 1

The following formula extender is prepared:

| Material | Weight % |
|---|---|
| Sequence 1 | |
| Dimethicone/Dimethicone PEG-10/15 crosspolymer | 7.00 |
| Dimethicone/dimethicone/vinyl dimethicone crosspolymer | 7.00 |
| PEG-9 Polydimethylsiloxyethyl dimethicone | 0.50 |
| Dimethicone | 14.50 |
| Silica/ethylene/methacrylate copolymer/isopropyl | 2.50 |
| titanium triisostearate | |
| Caprylyl glycol/phenoxyethanol/hexylene glycol | 0.70 |
| Sequence 2: | |
| Purified water | 33.10 |
| Sequence 3 | |
| Disodium EDTA | 0.07 |
| Sodium chloride | 1.00 |
| Sequence 4 | |
| Butylene glycol | 3.50 |
| Xanthan gum | 0.10 |
| Sequence 5 | |
| Purified water | 15.50 |
| Sequence 6 | |
| Disodium EDTA | 0.03 |
| Sequence 7 | |
| Butylene glycol | 4.50 |
| Sequence 8 | |
| Titanium dioxide/PEG-12 dimethicone | 10.00 |

Three additional compositions are prepared in which sequence 8 is either a red iron oxide, a yellow iron oxide or a black iron oxide.

The composition is prepared as follows. Sequences 2 and 3 are mixed together. The components of Sequence 4 are mixed on their own, then added to 2 and 3. Sequence 6 is added to Sequence 5, mixed, then Sequence 7 is added to 5 and 6. Sequence 8 is added to the mixture of 5, 6 and 7 then mixed in a Silverson mixer, at about 100-300 rpm, to disperse the pigment throughout the mixture. Once that's complete, the Sequence 2 and 3 mixture is combined with Sequence 1, and to form the primary emulsion, and mixed for about 15 minutes at 2800-3400 rpm. The Sequence 5, 6, 7 and 8 mixture is added to the emulsion using a prop mixer, at a speed chosen to achieve the desired micelle size. To obtain micelles of about 5-15μ, mixing speed is about 1800 rpm under propeller mixing for about 10 minutes. Each individually pigmented emulsion is then combined with the others, one at a time, and mixed at 500-800 rpm with propeller agitation for about 5 minutes to produce the finished product.

### EXAMPLE 2

This example illustrates a powder composition of the invention

| Material | Weight % |
|---|---|
| Sequence 1 | |
| Talc | 30.97 |
| Magnesium myristate | 4.00 |
| Bismuth oxychloride | 10.00 |
| Calcium aluminum borosilicate | 10.00 |
| Polyethylene | 10.00 |
| Silica encapsulated red iron oxide | 0.60 |
| Silica encapsulated yellow iron oxide | 1.40 |
| Silica encapsulated black iron oxide | 0.33 |
| Mica | 23.00 |
| Polyethylene | 2.00 |
| Sequence 2 | |
| Octyldodecyl stearoyl stearate | 2.00 |
| Lecithin | 0.20 |
| Bis-diglyceryl polyacyladipate-2 | 0.70 |
| Sequence 3 | |
| Penterythrityl tetraoctanoate | 2.50 |
| Corn oil/retinyl palmitate | 0.10 |
| Linoleic acid | 0.20 |
| Phytosqualane/soybean phospholipid/phytosterol | 1.50 |
| Phenoxyethanol/isopropylparaben/isobutyl | 0.30 |
| paraben/butylparaben | |
| Fragrance | 0.20 |

Using an Osterizer, the Sequence 1 components are mixed gently until homogeneous. The binders (Sequence 2) are added drop by drop and mixed gently. The actives of Sequence 3 are then added. At this stage the powder can be pressed, or left loose. An alternative method of preparation is to prepare formula extenders of individual colors, which are then subsequently mixed together.

1. A color cosmetic composition containing at least two different pigment types and a carrier, the pigments of different types being physically separated from each other in individual capsules within the carrier.

2. The composition of claim 1 in which the capsule comprises a chemical shell encompassing the pigment.

3. The composition of claim 2 in which the capsule is a colored glass bead.

4. The composition of claim 2 in which the capsule is comprises a silica shell.

5. The composition of claim 2 in which the capsule is surface-treated.

6. The composition of claim 2 which is an anhydrous composition.

7. The composition of claim 6 which is a powder composition.

8. The composition of claim 6 in which the capsule comprises a silica shell.

9. The composition of claim 1 which is a water and oil emulsion.

10. The composition of claim 9 in which the capsule is a micelle.

11. A color cosmetic emulsion composition containing at least two different pigments and at least two dispersed micellar phases, in which each different pigment is contained within micelles in a different dispersed phase within the carrier.

12. The composition of claim 11 in which the micelles have an average size of from about 1 to about 400μ.

13. The composition of claim 12, in which the micelles have an average size of less than about 100μ.

14. The composition of claim 12 in which the micelles have an average size of about 1 to about 60μ.

15. The composition of claim 12 in which the micelles have an average size of about 5 to about 20μ.

16. The composition of claim 12 in which the pigment is in an oil phase micelle, and is hydrophobically surface-treated.

17. The composition of claim 12 in which the pigment is in a water phase micelle and is hydrophilically surface-treated.

18. A method of making a color cosmetic composition containing at least two different pigments and a carrier, the method comprising the steps of (a) preparing one extender for each different pigment, and (b) blending each extender into the carrier without grinding the pigments together, whereby a composition is obtained in which there is substantially no physical contact in the carrier between the different types of pigments.

19. The method of claim 18 in which the composition is anhydrous and blending occurs at a speed of about 500 to about 800 rpm.

20. The method of claim 18 in which the composition is a water and oil emulsion and the blending occurs at a speed of about 500 to about 1200 rpm.

21. A composition obtained according to the method of claim 18.

22. A composition obtained according to the method of claim 19.

23. The composition of claim 22 which is a powder composition.

24. A composition obtained according to the method of claim 20.

25. A method of making a color cosmetic composition which comprises blending into a carrier at least two different pigments that are individually encapsulated in a chemical shell, and dispersing the pigments throughout the carrier.
